(19) United States
(12) Patent Application Publication
(10) Pub. No.: US 2009/0156987 A1
(43) Pub. Date: Jun. 18, 2009

(54) APPARATUS AND METHOD OF DELIVERY OF SUBSTANCES INTO TARGET TISSUE

(76) Inventors: Barbara Wanamaker McLean, Somis, CA (US); Sherry Thomas, North Hollywood, CA (US)

Correspondence Address: MARVIN E. JACOBS, ESQ., 1767 MARISOL DRIVE, VENTURA, CA, 93001, US

(21) Appl. No.: 11/957,073

(22) Filed: Dec. 14, 2007

(51) Int. Cl. A61M 37/00

(52) U.S. Cl. 604/60

(57) ABSTRACT

A barrel having a sharp cutting end is inserted into an animal body to form a channel connected to a pocket in the target tissue. A piston head mounted in the barrel has a chamber housing a solid element which contains a substance for treating the target tissue. Means associated with the piston head such as a push rod received in the piston bore is utilized to expel the solid element from the chamber into the pocket. The treating substance such as a capsule containing a liquid, gel or other substance is released from the capsule and coats the target tissue in the pocket.

### TECHNICAL FIELD

This invention relates to a method for delivering substances into the body, and more particularly, the present invention relates to an instrument for delivering the substance to target tissue, preferably while encapsulated in a membrane selected to release the substance to the tissue area.

### BACKGROUND OF THE INVENTION

Current methods for delivery of substances into the body, either manually or surgically, are often ineffective or often require careful dosage measuring with each application. There is often a loss of the substance during delivery that results in inadequate dosing. Certain substances need to be precisely and quantitatively measured and delivered completely into body tissue. Actual delivery is often cumbersome, inaccurate, and messy. Delivery into certain cavities is difficult and/or often impossible to administer accurately.

Current delivery systems include:

Suppositories are usually inserted into the rectum or vagina. They cannot deliver a liquid, cream or gel effectively or at the correct volume. They may be slow to dissolve and are easily expelled by contractions of surrounding muscle, lack of sphincter control or by gravity before the full amount of the substance to be delivered is administered. Since it is impossible to determine how much of the substance was absorbed, another application of a suppository may result in under or overdosing. Because suppositories are in a partially solid or completely solid form, the dissolution time may be prolonged or not occur at all.

Hard tablets can irritate tissue, can be easily expelled, dissolve slowly before adequate absorption, or cause inconsistent coating of the directed tissue area.

Creams, gels and lotions are not only messy and cumbersome to administer, but often a significant portion of the dosage is lost and wasted on the delivery instrument and/or outside the cavity.

It is difficult to administer an accurate dosage of liquid to most body cavities and tissues. As with the creams and gels, they are often messy and cumbersome to administer, and often a portion of the dosage is lost and wasted on the delivery instrument and/or outside the cavity.

There is a need for accurate administration of a pre-measured substance into a cavity or tissue area, either manually or surgically, preferably with quick delivery to the target tissue.

As disclosed in my co pending application Ser. No. ______ filed concurrently herewith entitled “DISPENSING ENCAPSULATED FLUIDS, POWDERS OR MICROSPHERES, SUITABLY TIME RELEASED MICROSPHERES INTO BODY CAVITIES OR TISSUES” (Docket Number 517-20-001) the disclosure of which is completely and expressly incorporated herein by reference, capsules containing liquids, powders or micro spheres, suitably time released microspheres are described which can be inserted into body cavities. The membrane melts, dissipates or dissolves in the cavity to expel the liquid, powder or microsphere, suitably time released micro spheres, onto the walls of the cavity. Insertion of the capsules into natural cavities having exposed tissue such as vaginal, rectal, nasal, etc. tissue can be accomplished by digital insertion through the opening of the cavity. However, administration of capsules into internal tissue is not possible by manual, digital administration.

### STATEMENT OF THE INVENTION

In accordance with the invention, a cavity or pocket is formed by a tool which surgically cuts a channel through exterior tissue ending in a pocket or cavity in target tissue such as an organ or a growth either benign or cancerous. A piston head with a chamber formed in the distal face of the head is placed in the barrel of the tool. A cylindrical rod with a hollow bore is connected to the piston head. A capsule is placed in the chamber and a push rod is placed in the bore of the piston rod and pushed until the distal end of the rod traverses a passage through the head, pushes the capsule out of the chamber into the pocket. A central rod is mounted within the bore for axial movement and extends above the piston handle. The end of the rod is pushed to eject the capsules into the pocket. The rod may also be used to fracture the membrane of the capsule to release the encapsulated liquid. The inserter tool is then removed from the pocket and the channel.

The liquid is quickly released to treat exposed tissue. All of the dose of liquid is dispensed into the cavity and is quickly absorbed into the tissue.

These and other attendant advantages of the invention will become apparent in the following description when considered in conjunction with the accompanying drawings.

### BRIEF DESCRIPTION OF THE DRAWINGS

FIG. 1 is a schematic view of the outer barrel of an insertion instrument for forming a pocket in target tissue;

FIG. 2 is a schematic view of an insertion piston;

FIG. 3 is a schematic view of a piston rod;

FIG. 4 is a schematic view of the outer barrel of FIG. 1 with the cutting end inserted into target tissue to form a channel pocket; and

FIG. 4A is a schematic view of target tissue shown with the barrel removed;

FIG. 5 is a schematic view of the instrument of FIG. 2 shown with a piston and in the channel a capsule positioned in a chamber in the distal face of a piston;

FIG. 6 is a schematic view of an assembly unit with the distal end of the push rod forcing a capsule out of the chamber into the pocket;

FIG. 7 is a schematic view of the implanted capsule in the pocket after withdrawal of the instrument assembly;

FIG. 8 is a front view in elevation of a first embodiment of a capsule for delivering a liquid into a body cavity;

FIG. 9 is a front view in elevation of a second embodiment of capsule suitable for digital insertion;

FIG. 10 is a sectional view taken along line 10 - 10 of FIG. 9;

FIG. 11 is a view in section of a capsule within a capsule embodiment;

FIG. 12 is a schematic view of a capsule divided into two compartments;

FIG. 13 is a schematic view of an alternative version of a two compartment capsule;

FIG. 14 is a schematic view of a membrane encapsulated liquid suspension;

FIG. 15 is a schematic view of a capsule divided into four compartments by membrane walls.

### DESCRIPTION OF THE INVENTION

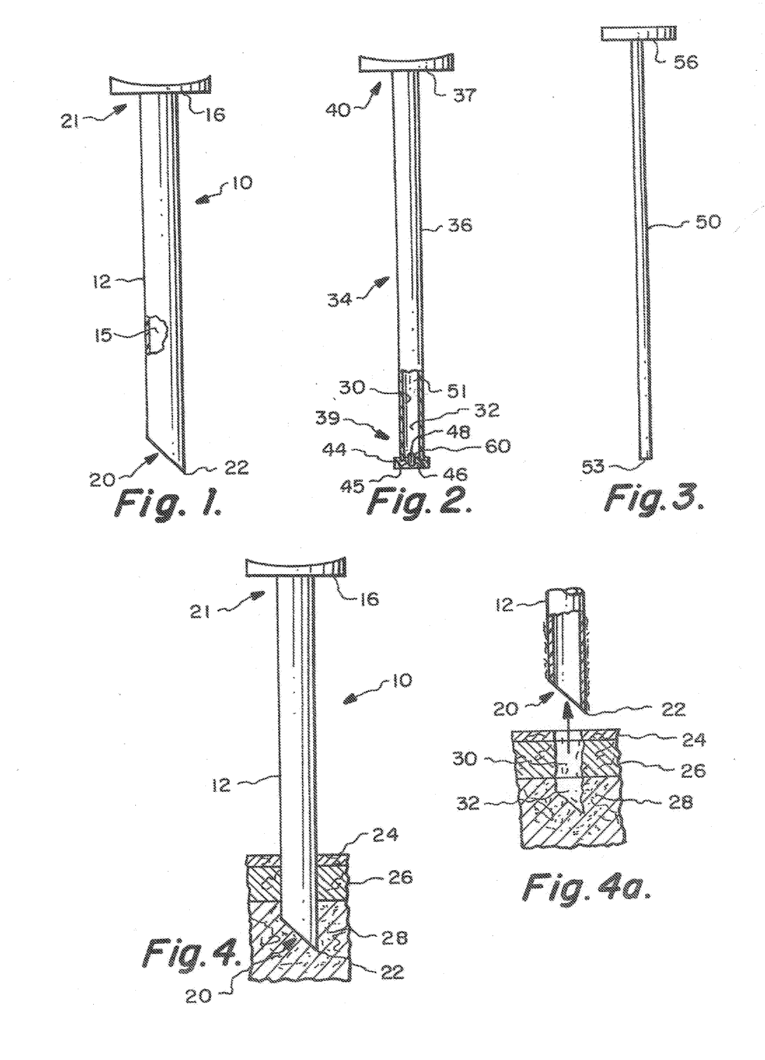

Referring now to FIGS. 1, 4, and 4A, the instrument 10 consists of a barrel 12. The distal end 20 of the barrel 12 terminates in a pointed, tissue cutting or burrowing tool end 22 similar to a biopsy needle. The other end 21 is fitted with a concave topped handle 16. Referring now to FIGS. 2,3,4 & 4a as the barrel 12 is pushed through a layer of skin 24 FIG. 4 and tissue 26 into target tissue such as a tumor 28, a channel 30 and pocket 32 are formed by the cutting tool 22.

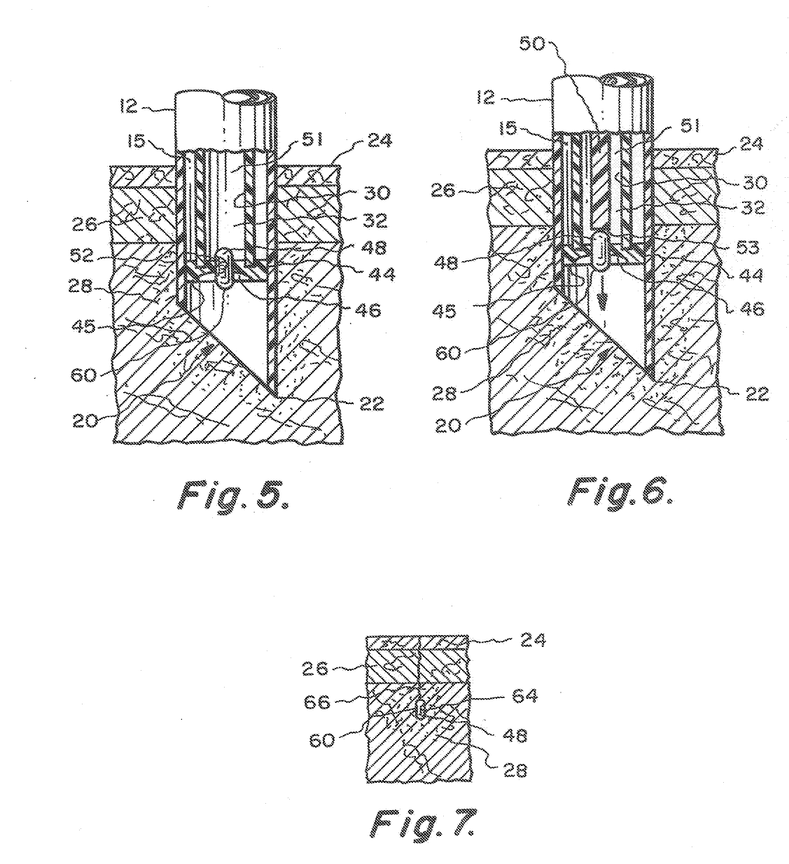

A dispensing piston 34 as shown in FIG. 2, including a hollow first rod 36 and a handle 37 is attached to the proximal end 40 end of the rod 36. The distal end 39 of the rod terminates in piston head 44. The distal face 45 of the piston head 44 is shaped to contain a detent forming a chamber 46 which holds a capsule 48. As shown in FIGS. 3, 5 and 6, a push rod 50 is downwardly mounted in the bore 51 of the rod 36. The distal end 53 of the rod 50 penetrates a slot 51 through the piston head 44. The push rod 50 extends into the channel 30 and pocket 32, FIG. 4a.

The proximal end of the push rod 50 is connected to a push handle 56 disposed a distance above the piston handles 16 and 37 necessary to traverse the longitudinal length of the channel 30 and pocket 32.

Target tissue 28 such as an infected organ is treated by pushing the pointed end 22 of the barrel 12 through skin layer 24 then tissue layer 26 into a tumor 28 to form a pocket 32 and a channel 30 as shown in FIGS. 4 & 4a.

If necessary, an agent to control bleeding can be injected through the barrel 12 or bore of push rod 50 into the pocket 32. As shown in FIG. 2, a capsule 48 is then placed in the chamber 46 in the piston head 44. The piston head 44 is placed in the bore 15 of the barrel 12 and handle 16 is manually pushed until the head 44 enters the pocket 32. The push rod 50 is then activated by pressing the push handle 56 to eject the capsule 48 into the pocket 32. The membrane 60 of the capsule 48 fragments, melts, dissolves or dissipates in the pocket 32 to release the encapsulated liquid 52 which is released into the target tissue area 66 of pocket 32. The instrument 10 is then withdrawn from the body.

The capsule utilized with the insertion instrument of the invention can contain any solid material or a dissolving solid, liquid, powder, microspheres, cream or gel encapsulated in a membrane that fragments, melts, dissolves or dissipates in the cavity or pocket formed by the instrument. The solid capsule or encapsulated material can contain a substance treatment unit dosage amount of a medicine suitable to treat and/or cure the affected condition of the target tissue.

The outer membrane can take various shapes and/or sizes to accommodate the specific need of a substance to be delivered or to accommodate the specific body cavity proportions.

The membrane may be layered to contain more than one substance for delivery. Outer membrane will melt/dissolve/dissipate to release the first substance, and then the next membrane will melt to release a subsequent substance. This second substance or dosage may be a suspension within the encapsulated liquid or may be a part of a partitioned module.

Various thicknesses, dissipating temperatures, or chemical compositions of the membrane may be used to vary dissipation timing.

The specific chemistry of the membrane may have to be altered or coated with an outside layer to prevent premature reactions with substances present in the cavity or entrance to the cavity.

Capsules may be produced with contents targeting other problems including, but not limited to:
a) Cavity pH balance
b) Candida control
c) Birth control
d) Tissue health
e) Hormone balance
f) Treatment of sexually transmitted diseases
g) Lactobacillus homeo stasis
h) Treatment of cancerous target tissue
i) Treatment of other medical conditions

The membrane capsule can be produced empty. The physician or pharmacist may inject a therapeutic liquid, gel, powder or other substance into the capsule.

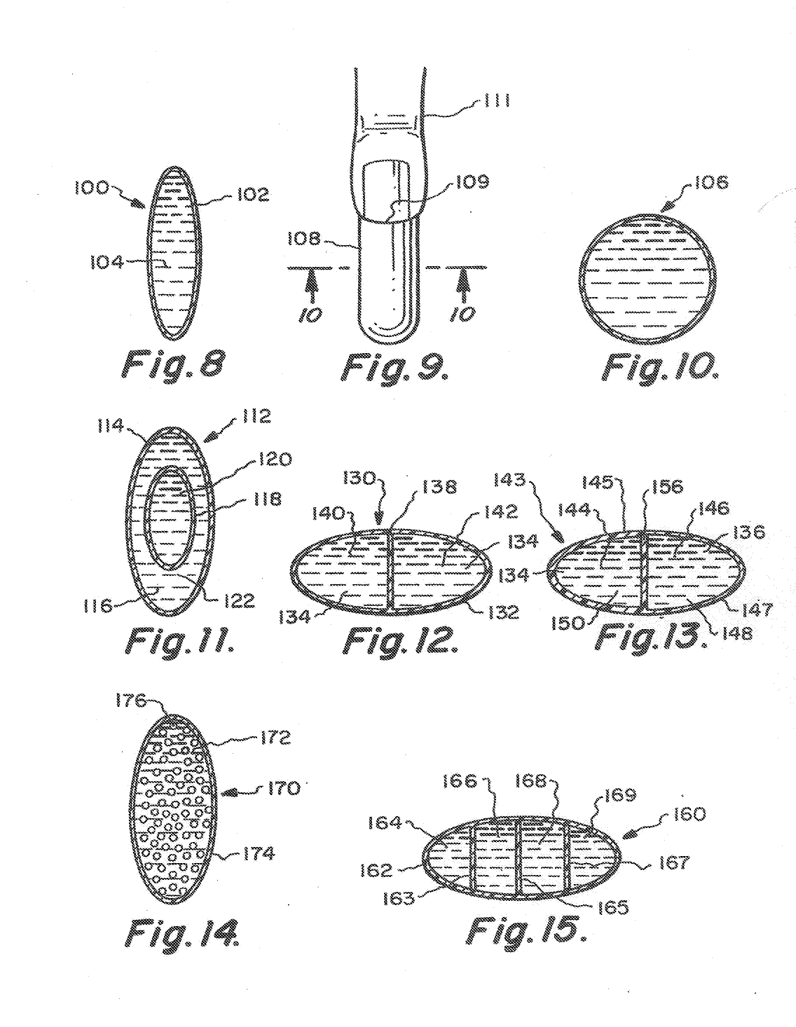

Referring now to FIG. 8, a capsule 100 can take the form of an elongated, thin, oval membrane 102 encapsulating a body 104 of liquid. FIGS. 9 and 10 illustrate a wide capsule 106 having a convex distal end 108 and a concave proximal end 109 in which the top of a finger 111 can be placed during insertion of the capsule 106. The distal end 108 can be conically shaped to aid in insertion.

FIG. 11 illustrates a capsule 112 having an outer membrane skin 114 which encapsulates a first body of liquid 116 and an inner membrane and film 118 which encapsulates a second body of liquid 120. The liquids 116, 120 can be the same and delivery will occur in stages allowing the push rod 50 to breach the outer membrane skin 114 to expel the first body of liquid 116 into tissue, and after absorption the push rod 50 is again pressed to rupture the inner capsule 122 which then expels the second body of liquid 120. The liquids 116 and 120 can be different such as liquid 116 controlling blood flow and liquid 120 containing a therapeutic substance.

FIG. 12 illustrates a two compartment capsule 130 in which the interior of an oval-shaped membrane 132 is partitioned into two chambers 134 and 136 by a wall 138. The liquids 140, 142 contained within chambers 134 and 136 will be dispensed simultaneously on breach of the outer membrane film 132 or in sequence if the capsule 130 is positioned with the membrane wall 138 normal to the push rod 50. The push rod 50 can be advanced stepwise to sequentially disrupt the membranes enclosing chambers 134, 136 Release may occur with membrane melting and sequentially release the bodies of liquid 140, 142.

In FIG. 13 the first chamber 139 has a thicker membrane 145 than the membrane 147 surrounding the second chamber 149. The bodies of liquids 144, 146 are contained within compartments 148, 150. However, the thickness of the outer membrane 145 connected to a partition wall 156 is thicker than the other portion the membrane 147 also connected to the partition wall 156. The thinner membrane 147 will be breached first and deliver the contained liquid 146 to targeted tissue. The thicker membrane 145 will breach or dissipate at a later time.

In FIG. 15 a capsule 160 having an outer membrane 162 is partitioned into 4 chambers 164, 166, 168, 169 by means of walls 163, 165, 167 which extend across the membrane 162. The membrane walls can also be disposed parallel to the central axis of the capsule.

FIG. 14 shows a capsule 170 in which the liquid suspension or powders 172 encapsulated by the membrane 174 contains a dispersion of microspheres or other particles 176.

It is to be realized that the preceding description is for the purpose of enabling one to understand the invention and is not intended to limit the invention, except as defined in the following claims.

1. A method of treating an animal having diseased internal target tissue comprising the steps of:
forming a channel through skin and tissue and forming a pocket in the target tissue, the channel and pocket being in communication; and the pocket having walls;
inserting a module containing a treating substance for the target tissue through said channel into the pocket;
releasing said substance from the module element; and depositing the substance into the target tissue or cavity.

2. A method according to claim 1 in which the channel and pocket are formed by means of a barrel having a cutting or burrowing end and cutting a channel through the skin and tissue with the cutting end until a pocket is formed in the target tissue.

3. A method according to claim 2 in which the element is disposed in the piston head, the piston head being disposed in the barrel.

4. A method according to claim 2 in which the piston head is connected to one end of a hollow piston rod, a handle having a bore is connected to the other end of the piston rod, a push rod being disposed within the bore of the piston rod;
moving the piston rod until the piston head is within the pocket; and
displacing the solid element from the piston head into the pocket by movement of the push rod into the piston head.

5. An instrument assembly for placing a solid treating element into target tissue comprising in combination:
A barrel having a sharp end for cutting through tissue of an animal subject and for forming a pocket in target tissue, said barrel having a second distal end;
A piston having a head downwardly mounted in the barrel;
A hollow piston rod extending from the piston head to a proximal position above the second end of the barrel;
A push rod mounted for axial translation in the hollow piston rod, said push rod having a distal end extending into the piston head and a proximal end extending above the distal position of the end of the piston rod;
means associated with the piston head for holding the solid, treating element; and
means associated with the push rod for expelling the solid treating element from the piston head into the pocket within the target tissue.

6. An instrument according to claim 5 in which a first pushing means is connected to the proximal second end of the barrel.

7. An instrument assembly according to claim 6 in which a top member is attached to an end of the piston rod at the proximal position.

8. An instrument assembly according to claim 7 in which a top is attached to the proximal end of the push rod and the length of the push rod exceeds the length of the piston rod.

9. An instrument assembly according to claim 8 in which the push rod is hollow for direct injection of treatment fluid into target tissue while the piston rod and barrel are disposed in the target tissue.
